(19) United States
(12) Patent Application Publication
(10) Pub. No.: US 2009/0203695 A1
(43) Pub. Date: Aug. 13, 2009

(54) Compounds IV

(75) Inventors: Michael Higginbottom, Cambridgeshire (GB); Viet-Anh Anne Horgan (nee Nguyen), Surrey (GB); Iain Simpson, Cambridge (GB)

Correspondence Address: FISH & RICHARDSON PC, P.O. BOX 1022, MINNEAPOLIS, MN, 55440-1022, US

(73) Assignee: Biovitrum AB, Stockholm (SE)

(21) Appl. No.: 12/315,830

(22) Filed: Dec. 5, 2008

Related U.S. Application Data
(60) Provisional application No. 61/022,966, filed on Jan. 23, 2008.

(30) Foreign Application Priority Data
Dec. 5, 2007 (SE) 0702699-0

(51) Int. Cl. A61K 31/5377; C07D 413/02; A61P 3/04; A61P 3/10; A61P 15/08; A61P 37/00; A61P 17/02; A61P 29/00; A61P 9/00

(52) U.S. Cl. 514/235.8; 544/129; 514/237.2; 544/121

(57) ABSTRACT

The present application relates to new compounds of formula (I),

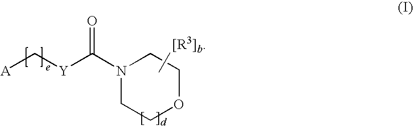

to pharmaceutical compositions comprising the compounds, to processes for their preparation, and to the use of the compounds as leptin receptor modulator mimetics in the preparation of medicaments against conditions associated with weight gain, type 2 diabetes and dyslipidemias.

### CROSS-REFERENCE TO RELATED APPLICATIONS

This application claims the benefit of Swedish Application No. 0702699-0, filed Dec. 5, 2007 and of U.S. Provisional Application No. 61/022,966, filed Jan. 23, 2008, the entire disclosures of which are hereby incorporated by reference in their entireties.

### FIELD OF THE INVENTION

The present application relates to new morpholine derivatives, to pharmaceutical compositions comprising the compounds, to processes for their preparation, and to the use of the compounds as leptin receptor modulator mimetics in the preparation of medicaments against conditions associated with weight gain, type 2 diabetes and dyslipidemias.

### BACKGROUND ART

The prevalence of obesity is increasing in the industrialized world. Typically, the first line of treatment is to offer diet and life style advice to patients, such as reducing the fat content of their diet and increasing their physical activity. However, some patients may also need to undergo drug therapy to maintain the beneficial results obtained from adapting the aforementioned diet and lifestyle changes.

Leptin is a hormone synthesized in fat cells that is believed to act in the hypothalamus to reduce food intake and body weight (see, e.g., Bryson, J. M. (2000) Diabetes, Obesity and Metabolism 2: 83-89).

It has been shown that in obese humans the ratio of leptin in the cerebrospinal fluid to that of circulating leptin is decreased (Koistinen et al., (1998) Eur. J. Clin. Invest. 28: 894-897). This suggests that the capacity for leptin transport into the brain is deficient in the obese state. Indeed, in animal models of obesity (NZO mouse and Koletsky rat), defects in leptin transport have been shown to result in reduced brain leptin content (Kastin, A. J. (1999) Peptides 20: 1449-1453; Banks, W. A. et al., (2002) Brain Res. 950: 130-136). In studies involving dietary-induced obese rodents (a rodent model that is believed to more closely resemble human obesity, see, e.g., Van Heek et al. (1997) J. Clin. Invest, 99: 385-390), excess leptin administered peripherally was shown to be ineffective in reducing food intake and body weight, whereas leptin injected directly into the brain was effective in reducing food intake and body weight. It has also been shown that in obese humans with excess circulating leptin, the signaling system became desensitized to the continual stimulation of the leptin receptors (Mantzoros, C. S. (1999) Ann. Intern. Med. 130: 671-680).

Amgen has conducted clinical trials with recombinant methionyl human leptin. The results from these trials were mixed, as even in the presence of high plasma concentrations of leptin weight loss was variable, and the average weight reduction in the cohort of patients tested relatively small (Obesity Strategic Perspective, Datamonitor, 2001).

Several attempts at finding active fragments have been reported in the literature since the discovery of the leptin gene coding sequence. An example is by Samson et al. (1996) Endocrinol. 137: 5182-5185 which describes an active fragment at the N-terminal (22 to 56). This sequence was shown to reduce food intake when injected ICV whereas a sequence taken at the C-terminal was shown not to have any effect. Leptin fragments are also disclosed in International Patent Application WO 97/46585.

Other reports looking at the C-terminus part of the sequence reported a possible stimulation of luteinising hormone production by a 116-130 fragment (Gonzalez et al., (1999) Neuroendocrinology 70:213-220) and an effect on GH production following GHRH administration (fragment 126-140) (Hanew (2003) Eur. J. Endocrin. 149: 407-412). Leptin has recently been associated with inflammation. It has been reported that circulating leptin levels rise during bacterial infection and in inflammation (see Otero, M et al. (2005) FEBS Lett. 579: 295-301 and references therein). Leptin can also act to increase inflammation by enhancing the release of pro-inflammatory cytokines TNF and IL-6 from inflammatory cells (Zarkesh-Esfahani, H. et al. (2001) J. Immunol. 167: 4593-4599). These agents in turn can contribute to the insulin resistance commonly seen in obese patients by reducing the efficacy of insulin receptor signaling (Lyon, C. J. et al. (2003) Endocrinol. 44: 2195-2200). Continuous low grade inflammation is believed to be associated with obesity (in the presence and absence of insulin resistance and Type II diabetes) (Browning et al. (2004) Metabolism 53: 899-903, Inflammatory markers elevated in blood of obese women; Mangge et al. (2004) Exp. Clin. Endocrinol. Diabetes 112: 378-382, Juvenile obesity correlates with serum inflammatory marker C-reactive protein; Maachi et al. (2004) Int. J. Obes. Relat. Metab. Disord. 28: 993-997, Systemic low grade inflammation in obese people). Leptin has also been implicated in the process of atherogenesis, by promoting lipid uptake into macrophages and endothelial dysfunction, thus promoting the formation of atherosclerotic plaques (see Lyon, C. J. et al. (2003) Endocrinol. 144: 2195-2200).

Leptin has also been shown to promote the formation of new blood vessels (angiogenesis) a process implicated in the growth of adipose tissue (Bouloumie A, et al. (1998) Circ. Res. 83: 1059-1066). Angiogenesis has also been implicated in diabetic retinopathy (Suganami, E. et al. (2004) Diabetes. 53: 2443-2448).

Angiogenesis is also believed to be involved with the growth of new blood vessels that feed abnormal tumour cells. Elevated leptin levels have been associated with a number of cancers, in particular breast, prostate and gastrointestinal cancers in humans (Somasundar P. et al. (2004) J. Surg. Res. 116: 337-349).

Leptin receptor agonists may also be used in the manufacture of a medicament to promote wound healing (Gorden, P. and Gavrilova, O. (2003) Current Opinion in Pharmacology 3: 655-659).

Further, it has been shown that elevating leptin signaling in the brain may represent an approach for the treatment of depressive disorders (Lu, Xin-Yun et al. (2006) PNAS 103: 1593-1598).

### DISCLOSURE OF THE INVENTION

It has surprisingly been found that compounds of formula (I) are effective in reducing body weight and food intake in rodents. While not wishing to be bound by theory, it is proposed that the compounds of formula I modulate the leptin receptor signaling pathway.

In some embodiments, compounds with leptin receptor agonistic like properties can be useful for the treatment of disorders relating to leptin signaling, as well as conditions associated with weight gain, such as obesity. The inventors hypothesized that small molecule CNS penetrant leptin mimetics would be able to by-pass the limiting uptake system into the brain. Further, assuming that this situation mirrors the human obese condition, the inventors believe that a CNS-active leptinoid with a relatively long duration of action would make an effective therapy for the obese state and its attendant complications, in particular (but not limited to) diabetes.

In other embodiments, compounds with leptin receptor antagonistic like properties could be useful for the treatment of inflammation, atherosclerosis, diabetic retinopathy and nephropathy.

In a first aspect, the disclosure relates to a compound of formula (I),

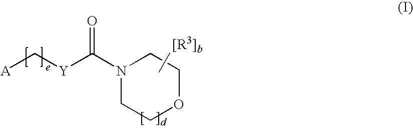

and pharmaceutically acceptable salts, hydrates, geometrical isomers, racemates, tautomers, optical isomers or N-oxides thereof, wherein:

A is selected from

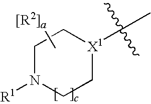

wherein X¹ is N or CH;
or

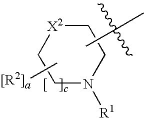

wherein X² is N(R¹), CH(R²) or O;

R¹ is selected from hydrogen, C₁₋₆-alkyl (unsubstituted or optionally substituted with one or more substituents independently selected from halogen, hydroxy, cyano and C₁₋₆-alkoxy), C₁₋₆-acyl (unsubstituted or optionally substituted with one or more substituents independently selected from halogen, hydroxy and C₁₋₆-alkoxy), phenyl and benzyl (both unsubstituted or optionally substituted with one or more substituents independently selected from halogen, hydroxy, cyano, nitro, CF₃, C₁₋₆-alkyl and C₁₋₆-alkoxy);

R² and R³ are independently selected from hydrogen, halogen, hydroxy, C₁₋₆-alkyl (unsubstituted or optionally substituted with one or more substituents independently selected from halogen, hydroxy and C₁₋₆-alkoxy) and C₁₋₆-alkoxy (unsubstituted or optionally substituted with one or more substituents independently selected from halogen, hydroxy and C₁₋₆-alkoxy);

Y is CH₂, O or N(R⁴);

R⁴ is hydrogen or C₁₋₄-alkyl;

a and b are each independently 1, 2 or 3;

c and d are each independently 0, 1 or 2; and

e is 1, 2 or 3;

Is with the proviso that the compound is not selected from the group consisting of:
2-(4-piperidinyl)ethyl (2R,6S)-2,6-dimethylmorpholine-4-carboxylate;
N-(4-piperidinylmethyl)-4-morpholinecarboxamide;
4-[1-oxo-3-(4-piperidinyl)propyl]-morpholine;
4-[1-oxo-3-(1-piperazinyl)propyl]-morpholine;
4-[3-[4-(4-chlorophenyl)-1-piperazinyl]-1-oxopropyl]-morpholine;
N-[3-(1-piperazinyl)propyl]-4-morpholinecarboxamide;
2-morpholinylmethyl 2-(hydroxymethyl)-4-morpholinecarboxylate; and
N-[[4-(3,4-dichlorophenyl)methyl]-2-morpholinyl]methyl-4-morpholinecarboxamide.

In a preferred embodiment, Y is O.

In another preferred embodiment, A is

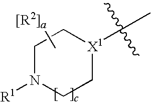

R¹ is preferably selected from hydrogen, C₁₋₄-alkyl, cyano-C₁₋₄-alkyl, phenyl and benzyl. In a most preferred embodiment, R¹ is hydrogen, methyl, cyanomethyl or benzyl.

R² and R³ are preferably independently selected from hydrogen and C₁₋₄-alkyl.

In a most preferred embodiment, R² and R³ are each independently hydrogen or methyl.

Particular preferred compounds of formula (I) are the compounds of formula (I′)

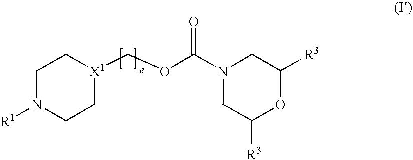

wherein X¹, R¹ and R³ and e are as defined in formula (I).

In preferred compounds of formula (I′), both R³ are methyl. It is especially preferred that the relative configuration of the two methyl groups is cis.

Specific preferred compounds of formula (I) are those selected from the group consisting of:
(1-benzylpiperidin-4-yl)methyl morpholine-4-carboxylate;
2-(4-methylpiperazin-1-yl)ethyl (2R,6S)-2,6-dimethylmorpholine-4-carboxylate; and
2-[4-(cyanomethyl)piperazin-1-yl]ethyl (2R,6S)-2,6-dimethylmorpholine-4-carboxylate.

Another aspect of the present disclosure is a compound of formula (I) for use in therapy.

In a further aspect, the disclosure relates to a compound of formula (I) for use in the treatment or prevention of any of the disorders or conditions described herein.

In yet a further aspect, the invention relates to the use of a compound of formula (I) in the manufacture of a medicament for the treatment or prevention of any of the disorders or conditions described herein.

In some embodiments, said compounds may be used in the manufacture of a medicament for the treatment or prevention of a condition that is prevented, treated, or ameliorated by selective action on the leptin receptor.

In some embodiments, said compounds may be used in the manufacture of a medicament for the treatment or prevention of conditions (in particular, metabolic conditions) that are associated with weight gain. Conditions associated with weight gain include diseases, disorders, or other conditions that have an increased incidence in obese or overweight subjects. Examples include: lipodystrophy, HIV lipodystrophy, diabetes (type 2), insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain, macular degeneration. In some embodiments, the compounds may also be used in the manufacture of a medicament for maintaining weight loss of a subject.

In some embodiments, compounds of formula (I) which are leptin receptor agonist mimetics may also be used in the manufacture of a medicament to promote wound healing.

In some embodiments, compounds of formula (I) which are leptin receptor agonist mimetics may also be used in the manufacture of a medicament for the treatment or prevention of conditions that cause a decrease in circulating leptin concentrations, and the consequent malfunction of the immune and reproductive systems. Examples of such conditions and malfunctions include severe weight loss, dysmenorrhea, amenorrhea, female infertility, immunodeficiency and conditions associated with low testosterone levels.

In some embodiments, compounds of formula (I) which are leptin receptor agonist mimetics may also be used in the manufacture of a medicament for the treatment or prevention of conditions caused as a result of leptin deficiency, or a leptin or leptin receptor mutation.

In some other embodiments, compounds of formula (I) which are leptin receptor antagonist mimetics may be used for the treatment or prevention of inflammatory conditions or diseases, low level inflammation associated with obesity and excess plasma leptin and in reducing other complications associated with obesity including atherosclerosis, and for the correction of insulin resistance seen in Metabolic Syndrome and diabetes.

In some embodiments, compounds of formula (I) which are leptin receptor antagonist mimetics can be used for the treatment or prevention of inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type 1 diabetes mellitus including the destruction of pancreatic islets leading to diabetes and the inflammatory consequences of diabetes); autoimmune damage (including multiple sclerosis, Guillam Barre Syndrome, myasthenia gravis); cardiovascular conditions associated with poor tissue perfusion and inflammation (such as atheromas, atherosclerosis, stroke, ischaemia-reperfusion injury, claudication, spinal cord injury, congestive heart failure, vasculitis, haemorrhagic shock, vasospasm following subarachnoid hemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), arthritis (such as rheumatoid arthritis, osteoarthritis, rheumatoid spondylitis, gouty arthritis), fibrosis (for example of the lung, skin and liver), multiple sclerosis, sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of the bowel (including Crohn's disease, ulcerative colitis and pyresis, irritable bowel syndrome, inflammatory bowel disease); HIV (particularly HIV infection), cerebral malaria, bacterial meningitis, osteoporosis and other bone resorption diseases, osteoarthritis, infertility from, endometriosis, fever and myalgia due to infection, and other conditions mediated by excessive anti-inflammatory cell (including neutrophil, eosinophil, macrophage and T-cell) activity.

In some embodiments, compounds of formula (I) which are leptin receptor antagonists mimetics may be used for the treatment or prevention of macro or micro vascular complications of type 1 or 2 diabetes, retinopathy, nephropathy, autonomic neuropathy, or blood vessel damage caused by ischaemia or atherosclerosis.

In some embodiments, compounds of formula (I) which are leptin receptor antagonist mimetics may be used to inhibit angiogenesis. Compounds that inhibit angiogenesis may be used for the treatment or prevention of obesity or complications associated with obesity. Compounds that inhibit angiogenesis may be used for the treatment or prevention of complications associated with inflammation diabetic retinopathy, or tumour growth particularly in breast, prostate or gastrointestinal cancer.

In a further aspect, the disclosure relates to a method for the treatment or prevention of any of the disorders or conditions described herein, which includes administering to a subject (e.g., a subject in need thereof, e.g., a mammal) an effective amount of a compound of formula I.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the disclosure provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this disclosure; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the disclosure is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabeling/assay techniques, blotting/chemiluminescence methods, real-time PCR, and the like.

In some embodiments, it may be advantageous if a compound of formula (I) is able to penetrate the central nervous system. In other embodiments, it may be advantageous if a compound of formula (I) is not able to penetrate the CNS. In general, it is expected that compounds that are leptin receptor agonist mimetics may be particularly useful for the treatment or prevention of obesity, insulin resistance, or diabetes (particularly glucose intolerance) if these compounds can penetrate the CNS. A person of ordinary skill in the art can readily determine whether a compound can penetrate the CNS. A suitable method that may be used is described in the Biological Methods section.

A leptin receptor response may be measured in any suitable way. In vitro, this may be done be measuring leptin receptor signaling. For example, phosphorylation of Akt, STAT3, STAT5, MAPK, shp2 or the leptin receptor in response to binding of leptin or a compound of the disclosure to the leptin receptor may be measured. The extent of phosphorylation of Akt, STAT3, STAT5, MAPK, shp2 or the leptin receptor may be determined for example by Western blotting or by ELISA. Alternatively, a STAT reporter assay may be used, for example STAT driven luciferase expression. A cell line expressing the leptin receptor may be used for such assays. In vivo, leptin receptor response may be measured by determining the reduction in food intake and body weight after administration of leptin or a compound of the disclosure.

The Biological Methods below describe assays and methods that can be used to determine whether a compound of the disclosure is a leptin receptor agonist mimetic or a leptin receptor antagonist mimetic.

A compound of formula (I) may be administered with or without other therapeutic agents. For example, where it is desired to reduce inflammation, the compound may be administered with an anti-inflammatory agent (for example, disease modifying anti-rheumatic drugs such as methotrexate, sulphasalazine and cytokine inactivating agents, steroids, NSAIDs, cannabinoids, tachykinin modulators, or bradykinin modulators). Where it is desired to provide an anti-tumour effect, a compound of formula (I) may be administered with a cytotoxic agent (for example, methotrexate, cyclophosphamide) or another anti-tumour drug.

Compounds of formula (I) may be radiolabeled (for example with tritium or radioactive iodine) for in vitro or in vivo applications, such as receptor displacement studies or receptor imaging.

A further aspect of the present disclosure relates to processes for the manufacture of compounds of formula (I) as defined above. In one embodiment, the process comprises:

(a) reacting a compound of formula (II):

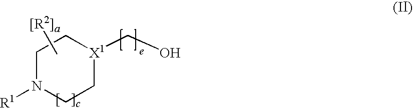

wherein X¹, R¹, R², a, c and e are as defined in formula (I),

with 4-nitrophenyl chloroformate or bis-(4-nitrophenyl)carbonate in the presence of a suitable base (such as NMM) in a suitable solvent (such as DCM), at −10 to 40° C., to form a compound of formula (III):

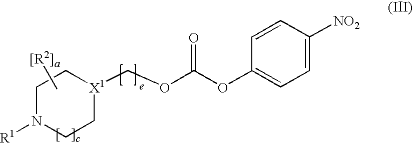

(b) reacting the compound of formula (III) with a compound of formula (IV):

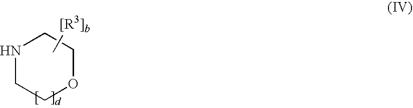

wherein R³, b and d are as defined in formula (I),
in the presence of a suitable base, (such as DIPEA or DMAP), in a suitable solvent (such as DMF), at −10 to 40° C., to obtain a compound of formula (I); and
(c) optionally, in one or several steps transforming a compound of formula (I) into another compound of formula (I).

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, iso-propyl, n-butyl, iso-butyl, sec-butyl, t-butyl, and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc.

Unless otherwise stated or indicated, the term “C₁₋₆-acyl” denotes a carbonyl group that is attached through its carbon atom to a hydrogen atom (i.e., a formyl group) or to a straight or branched C₁₋₅-alkyl group, where alkyl is defined as above. Examples of said C₁₋₆-acyl include formyl, acetyl, propionyl, n-butyryl, 2-methylpropionyl and n-pentoyl. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc. If a C₁₋₆-acyl group is optionally substituted with one or more substituents independently selected from halogen, hydroxy and C₁₋₆-alkoxy, said substituent can not be attached to the carbonyl carbon atom.

Unless otherwise stated or indicated, the term “C₁₋₆-alkoxy” denotes a straight or branched alkoxy group having from 1 to 6 carbon atoms. Examples of said C₁₋₆-alkoxy include methoxy, ethoxy, n-propoxy, iso-propoxy, n-butoxy, iso-butoxy, sec-butoxy, t-butoxy, and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

“Halogen” refers to fluorine, chlorine, bromine or iodine.

“Hydroxy” refers to the —OH radical.

“Nitro” refers to the —NO₂ radical.

“Cyano” refers to the —CN radical.

“Optional” or “optionally” means that the subsequently described event or circumstance may but need not occur, and that the description includes instances where the event or circumstance occurs and instances in which it does not.

The term “mammal” includes organisms, which include mice, rats, cows, sheep, pigs, rabbits, goats, and horses, monkeys, dogs, cats, and preferably humans. The subject may be a human subject or a non human animal, particularly a domesticated animal, such as a dog. “Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of formula (I). A prodrug may be inactive when administered to a subject in need thereof, but is converted in viva to an active compound of formula (I). Prodrugs are typically rapidly transformed in vivo to yield the parent compound, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs may be prepared by modifying functional groups, such as a hydroxy, amino or mercapto groups, present in a compound of formula (I) in such a way that the modifications are cleaved, either in routine manipulation or in vivo, to the parent compound. Examples of prodrugs include, but are not limited to, acetate, formate and succinate derivatives of hydroxy functional groups or phenyl carbamate derivatives of amino functional groups.

Throughout the specification and the appended claims, a given chemical formula or name shall also encompass all solvates and hydrates thereof. Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### Compositions

For clinical use, the compounds of the disclosure are formulated into pharmaceutical formulations for various modes of administration. It will be appreciated that the compounds may be administered together with a physiologically acceptable carrier, excipient, or diluent. The pharmaceutical compositions may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the disclosure may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Preparation of Compounds of the Invention

The compounds of formula (I) above may be prepared by, or in analogy with, conventional methods. Formation of the central urethane or urea linker is the key synthetic step in preparing the compounds formula (I). A large number of activating reagents can be used for the formation of a urethane or urea linker e.g. phosgene to form chloroformate of alcohols, or carbonyldiimidazole (CDI) to form imidazole carboxylates. Typically the urethane linkers incorporated into compounds of formula (I) have been synthesized utilizing 4-nitrophenyl chloroformate or bis-(4-nitrophenyl)carbonate as the activating agent. The preparation of intermediates and compounds according to the examples of the present disclosure may in particular be illuminated by the following Scheme I. Definitions of variables in the structures in the schemes herein are commensurate with those of corresponding positions in the formulae delineated herein.

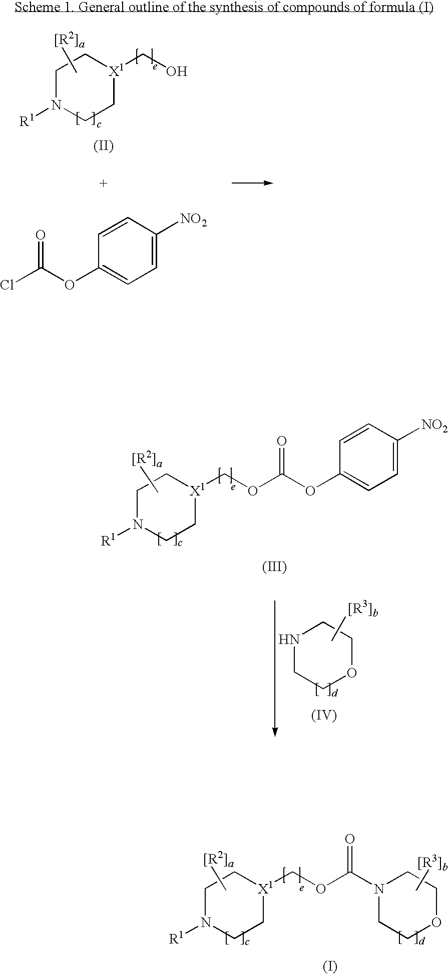

wherein X¹, R¹-R³ and a-e are as defined in formula (I)

Typically, the synthesis of compounds of formula (I) is performed by activation of the alcohol moiety. Treatment of alcohol (II) with 4-nitrophenyl chloroformate or bis-(4-nitrophenyl)carbonate in the presence of a base (such as NMM) yields the corresponding 4-nitrophenyl carbonate derivative (III). In the subsequent step, the activated carbonate (III) is treated with the appropriate morpholine derivative (IV) in the presence of a base (such as DIPEA or DMAP), to yield the desired compound of formula (I).

Alternatively, the morpholine derivative (IV) can be activated by treatment with 4-nitrophenyl chloroformate or bis-(4-nitrophenyl)carbonate in the presence of a base to form the corresponding carbamate derivative. The carbamate intermediate is then treated with the appropriate alcohol moiety (II) in the presence of a base to give the compound of formula (I).

The formation of the urethane is typically a two step process but this may also be performed in a one-pot reaction by formation of the activated intermediate in situ.

The necessary starting materials for preparing the compounds of formula (I) are either commercially available, or may be prepared methods known in the art.

The processes described below in the experimental section may be carried out to give a compound in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The following abbreviations have been used:

Boc tert-Butoxy carbonyl

DCM Dichloromethane

DIPEA N N-Diisopropylethylamine

DMAP N,N-Dimethylaminopyridine

DMF N,N-Dimethylformamide

ES⁺ Electrospray

Et₂O Diethyl ether

EtOAc Ethyl acetate

HIV Human immunodeficiency virus

HPLC High performance liquid chromatography

ICv Intracerebroventricular

LCMS Liquid Chromatography Mass Spectrometry

M Molar

[MH]⁺ Protonated molecular ion

NEt₃ Triethylamine

NMM N-methyl morpholine

RP Reverse Phase

tert Tertiary

TFA Trifluoroacetic acid

THF Tetrahydrofuran

Embodiments of the disclosure are described in the following examples with reference to the accompanying drawings, in which:

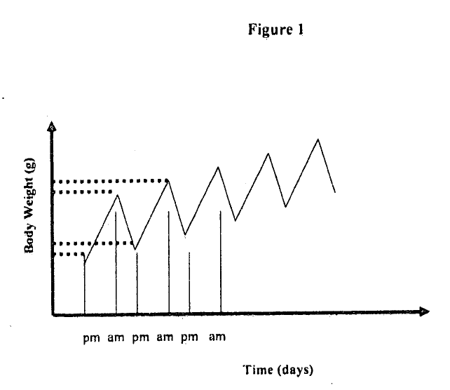

FIG. 1 is a schematic drawing illustrating weight gain and weight loss in mice during dark and light phases, respectively. The graph illustrates the large nocturnal weight increase versus the comparatively small body weight change over 24 hours

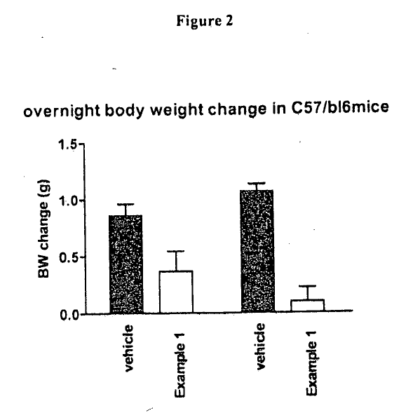

FIG. 2 shows the effect of Example 1 on the body weight in mice between the beginning of the dark phase and the beginning of the light phase (pm-am).

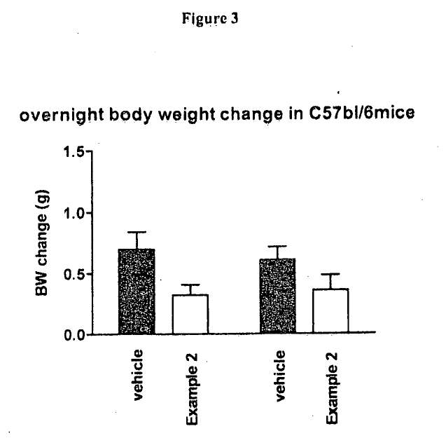

FIG. 3 shows the effect of Example 2 on the body weight in mice between the beginning of the dark phase and the beginning of the light phase (pm-am).

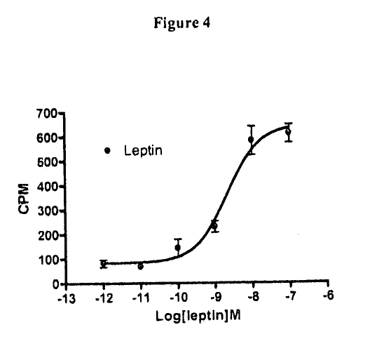

FIG. 4 shows the concentration-dependent increase in [³H]-thymidine incorporation by JEG-3 cells for leptin

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof.

The disclosure will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Commercially available anhydrous solvents were used for reactions conducted under inert atmosphere. Reagent grade solvents were used in all other cases, unless otherwise specified. Analytical LCMS was performed on a Waters ZQ mass spectrometer connected to an Agilent 1100 HPLC system. Analytical HPLC was performed on an Agilent 1100 system. Flash chromatography was performed on a Flash Master Personal system equipped with Strata SI-1 silica gigatubes. Reverse phase chromatography was performed on a Gilson system equipped with Merck LiChoprep® RP-18 (40-63 μm) 460×26 mm column, 30 mL/min, gradient of methanol in water. Preparative HPLC was performed on a Gilson system equipped with Phenomenex Hydro RP 15 0× 20 mm, 20 mL/min, gradient of acetonitrile in water. The compounds were automatically named using ACD 6.0 or 8.0.

Analytical HPLC data were obtained with:

System A: Phenomenex Synergi Hydro RP, (150×4.6 mm, 4 μm), gradient 5-100% CH₃CN (+0.085% TFA) in H₂O (+0.1% TFA), 1.5 mL/min, with a gradient time of 7 min, 200-300 nm, 30° C.

Analytical LCMS data were obtained with:

System B: Phenomenex Synergi Hydro RP (30×4.6 mm, 4 μm), gradient 5-100% CH₃CN (+0.085% TFA) in H₂O (+0.1% TFA), 1.5 mL/min, gradient time 1.75 min, 30° C.;

System C: Phenomenex Synergi Hydro RP, (150×4.6 mm, 4 μm), gradient 5-100% CH₃CN (+0.085% TFA) in H₂O (+0.1% TFA), 1 mL/min, gradient time 7 min, 30° C.; or

System D: Phenomenex Synergi Hydro RP, (150×4.6 mm, 4 μm), gradient 5-100% CH₃CN (+0.085% TFA) in H₂O (+0.1% TFA), 1 mL/min, gradient time 8 min, 25° C.;

### Example 1

### (1-Benzylpiperidin-4-yl)methyl morpholine-4-carboxylate Hydrochloride

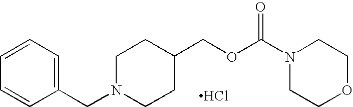

4-Piperidine methanol (5.49 g, 47.7 mmol) and NEt₃ (6.6 mL, 47.7 mmol) were dissolved in DCM (100 mL) and treated with benzoyl chloride (5.6 mL, 48 mmol). The reaction mixture was stirred for 18 hours, then washed sequentially with 2M aq HCl solution (2×200 mL) and 1M aq Na₂CO₃ solution (100 mL), dried (MgSO₄) and concentrated in vacuo to give (4-(hydroxymethyl)piperidin-1-yl)(phenyl)methanone as an orange oil which crystallised on standing. This solid was dissolved in THF (100 mL) under an argon atmosphere, cooled to 0° C. and treated with a 1M solution of LiAlH₄ in THF (50 mL, 50 mmol). The reaction mixture was allowed to warm to room temperature overnight, and then quenched by sequential addition of water, 1M aq NaOH solution and more water. The reaction mixture was stirred for a further 3 hours and then filtered through celite, and the filtrate was concentrated in vacuo to a volume of ca 40 mL and dried using two 20 g Isolute HM-N cartridges, eluting with EtOAc. The eluent was concentrated to give the intermediate (1-benzylpiperidin-4-yl)methanol (8.02 g, 82%) as a yellow oil.

(1-Benzylpiperidin-4-yl)methanol was dissolved in DCM (200 mL), treated with NMM (4.5 mL) and 4-nitrophenyl chloroformate (8.09 g, 40.1 mmol) and stirred for 18 hours. The reaction mixture was then washed with 1M aq Na₂CO₃ solution (200 mL) and sat aq NaHCO₃ solution (2×200 mL), dried (MgSO₄) and concentrated in vacuo to give (1-benzylpiperidin-4-yl)methyl 4-nitrophenyl carbonate (11.9 g, 82%) as a yellow solid.

A portion of (1-benzylpiperidin-4-yl)methyl 4-nitrophenyl carbonate (777 mg, 2.10 mmol) was dissolved in DMF (5 mL). NEt₃ (0.4 mL, 2.90 mmol), morpholine (0.30 mL, 3.41 mmol) and DMAP (10 mg) were added and the reaction mixture was stirred for 5 days before concentrating in vacuo. The residue was purified by reverse phase chromatography (gradient eluting with MeOH in water, with 1% formic acid in each solvent, 0-100%) followed by preparative HPLC (Phenomenex Hydro column, gradient eluting with CH₃CN in water, with 0-100%) to give a colourless gum, which was dissolved in DCM (5 mL), treated with 2M HCl in Et₂O (1 mL) and concentrated in vacuo to give (1-benzylpiperidin-4-yl)methyl morpholine-4-carboxylate hydrochloride (109 mg, 15%) as a white solid.

Analytical HPLC: purity 100% (System A, R_{T}=4.03 min); Analytical LCMS: purity 100% (System D, R_{T}=4.27 min), ES⁺: 319 [MH]⁺. HRMS calcd for C₁₈H₂₆N₂O₃: 318.1943, found 318.1956.

### Example 2

### 2-[4-(Cyanomethyl)piperazin-1-yl]ethyl (2R,6S)-2,6-dimethylmorpholine-4-carboxylate Formate

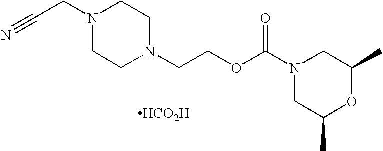

To a solution of 1-(2-hydroxyethyl)piperazine (51.7 g, 398 mmol) in DCM (500 mL) was added NEt₃ (70.0 mL, 526 mmol) and di-tert-butyl dicarbonate (80.0 g, 367 mmol). The reaction mixture was stirred overnight at room temperature and then washed with 1M aq Na₂CO₃ solution (2×300 mL), dried (MgSO₄) and concentrated in vacuo to give tert-butyl 4-(2-hydroxyethyl)piperazine-1-carboxylate (66.0 g, 72%) as a colourless oil.

Analytical LCMS: (System B, R_{T}=1.54 min), ES⁺: 231.4 [MH]⁺.

Bis(p-nitrophenyl)carbonate (1.52 g, 5.0 mmol) was dissolved in DCM (20 mL). The tert-butyl 4-(2-hydroxyethyl)piperazine-1-carboxylate from the previous step (1.15 g, 5.0 mmol) and NMM (0.55 mL, 5.0 mmol) were added and the reaction mixture was stirred at room temperature for 16 hours. The reaction mixture was diluted with DCM (40 mL) and washed with sat aq NaHCO₃ solution (5×50 mL), dried (Na₂SO₄) and concentrated in vacuo to give a yellow oil. The oil was purified by recrystallisation from EtOAc and heptane to give 2-(4-(tert-butoxycarbonyl)piperazin-1-yl)ethyl 4-nitrophenyl carbonate (1.208 g, 61%) as an orange solid.

Analytical LCMS: (System B, R_{T}=1.90 min), ES⁺: 396.5 [MH]⁺.

2-(4-(tert-Butoxycarbonyl)piperazin-1-yl)ethyl 4-nitrophenyl carbonate (0.868 g, 2.19 mmol) was dissolved in DMF (10 mL). cis-2,6-Dimethylmorpholine (0.284 mL, 2.3 mmol) and DIPEA (0.4 mL, 2.3 mmol) were added and the reaction mixture was stirred at room temperature for 48 hours and then concentrated in vacuo to give a yellow oil. The residue was dissolved in EtOAc (30 mL) and washed with 1M aq Na₂CO₃ solution (6×20 mL), dried (MgSO₄) and concentrated in vacuo to give tert-butyl 4-(2-(cis-2,6-dimethyl-morpholine-4-carboxyloyloxy)ethyl)piperazine-1-carboxylate (0.75 g, 92.5%) as a pale yellow oil.

Analytical LCMS: (System B, R_{T}=1.72 min), ES⁺: 372.5 [MH]⁺.

The tert-butyl 4-(2-(cis-2,6-dimethylmorpholine-4-carboxyloyloxy)ethyl)piperazine-1-carboxylate from the previous step (0.75 mg, ˜2 mmol) was dissolved in DCM (10 mL). TFA (2 mL) was added and the reaction mixture was stirred at room temperature for 18 hours and then concentrated in vacuo. The residue was dissolved in EtOAc (30 mL) with a few drops of DIPEA, then washed with sat aq NaHCO₃ solution (20 mL), dried (MgSO₄) and concentrated in vacuo to give cis-2-(piperazin-1-yl)ethyl 2,6-dimethylmorpholine-4-carboxylate as a yellow oil that was used without further purification.

cis-2-(piperazin-1-yl)ethyl 2,6-dimethylmorpholine-4-carboxylate was dissolved in THF (10 mL). DIPEA (0.521 mL, 3 mmol) and iodoacetonitrile (0.145 mL, 2 mmol) were added and the reaction mixture was stirred at room temperature for 16 hours and then concentrated in vacuo. The resulting residue was purified by normal phase column chromatography (gradient eluting with MeOH in DCM, 0-5%) followed by reverse phase column chromatography (gradient eluting with MeOH in water, with 1% formic acid in each solvent, 0-100%) to give 2-[4-(cyanomethyl)piperazin-1-yl]ethyl (2R,6S)-2,6-dimethylmorpholine-4-carboxylate formate (193 mg, 27%) as a colourless oil.

Analytical HPLC: purity 98.6% (system A, R_{T}=3.40 min); Analytical LCMS: purity 100% (System C, R_{T}=5.04 min), ES⁺: 311.5 [MH]⁺. HRMS calcd for C₁₅H₂₆N₄O₃: 310.2005, found 310.2017.

### Example 3

### 2-(4-Methylpiperazin-1-yl)ethyl (2R,6S)-2,6-dimethylmorpholine-4-carboxylate Formate

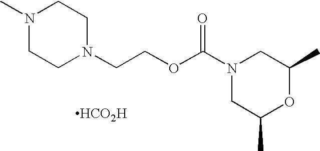

To a stirred solution of 1-(2-hydroxyethyl)piperazine (26 g, 0.2 mol) in DMF (200 mL) was added formic acid (752 mL, 0.2 mol) and formaldehyde (16.2 g, 0.2 mol, 37% solution in water). The reaction mixture was cautiously heated at 100° C. for 2 hours then stirred overnight at room temperature. The solvent was removed in vacuo. This procedure was repeated 3 further times to give ˜100 g of product. The crude products were combined and distilled under vacuum to give, at ˜74° C., 2-(4-methylpiperazin-1-yl)ethanol (51 g, 44%) as a colourless liquid.

Analytical LCMS: (System B, R_{T}=0.70 min), ES⁺: 145.1 [MH]⁺.

4-Nitrophenyl chloroformate (9.85 g, 49 mmol) was dissolved in DCM (200 mL), and cooled to 0° C. 2-(4-methylpiperazin-1-yl)ethanol from the previous step (7.2 g, 50 mmol) and NMM (6 mL) were added, and the reaction mixture allowed to warm gradually to room temperature over 16 hours. The reaction mixture was washed with 1M aq Na₂CO₃ solution. The organic phase was dried (MgSO₄), filtered and concentrated in vacuo to give 2-(4-methylpiperazin-1-yl)ethyl 4-nitrophenyl carbonate (10.7 g, 71%) as a yellow oil which solidified on standing.

Analytical LCMS: purity ˜80% (System B, R_{T}=1.70 min), ES⁺: 310.4 [MH]⁺.

2-(4-Methylpiperazin-1-yl)ethyl 4-nitrophenyl carbonate (3.00 g, 9.71 mmol) was dissolved in DMF (40 mL). DIPEA (1.77 mL, 10.2 mmol) and cis-2,6-dimethylmorpholine (1.25 mL, 10.2 mmol) were added and the reaction mixture was stirred at room temperature for 24 hours, and the reaction mixture was then concentrated in vacuo. The residue was dissolved in EtOAc (100 mL) and washed with a 1M aq Na₂CO₃ solution (7×50 mL). The organic phase was dried (MgSO₄) and concentrated in vacuo. The residue was purified by reverse phase chromatography (gradient eluting with MeOH in water, with 1% formic acid in each solvent, 0-100%) to give 2-(4-methylpiperazin-1-yl)ethyl (2R,6S)-2,6-dimethylmorpholine-4-carboxylate formate (1.09 g, 20%) as a colourless oil.

Analytical HPLC: purity 89.6% (System A, R_{T}=2.99 min); Analytical LCMS: purity 99% (System C, R₁=4.68 min), ES⁺: 286.5 [MH]⁻. HRMS calcd for C₁₄H₂₇N₃O₃: 285.2052, found 285.2063.

### Biological Tests

Measurement of Overnight Body Weight Change in Male C57 bl/6 Mice

This model studies the effects of compounds on body weight gain during the pm-am period in order to maximise the effective window. Typically the mice gain about 1 g in weight during the dark phase and then loose the majority of this weight gain during the light phase, as represented in FIG. 1. The weight difference over any 24 hour period is very small whilst the weight difference between the beginning of the dark phase and the beginning of the light phase (pm-am) is maximal.

It is important to measure body weight change over the dark phase. If mice are dosed with an active compound on two consecutive days and the bodyweight change is recorded 48 hours after the first dose then no significant effect is observed. However if the body weight change over the dark phase only is considered a significant and robust effect is seen. This is because the mice rebound during the light phase to compensate for the lack of weight gain over the dark phase. Very active long lasting compounds may also diminish this rebound and reduce the body weight over the 48 hours.

Weight Change Over Consecutive Days in C57 bl/6 Male Mice:

The weight difference between the beginning of the dark phase and the beginning of the light phase (pm-am) is greater than the weight difference measured between pm and pm on 2 consecutive days. The effect of the compounds on the pm-am difference was therefore studied in order to maximise the effect window.

C57 bl/6 mice were grouped (5 per cage) and left 5 days for acclimatisation. A single intraperitoneally (ip) administered dose (60 mg/kg) was given just prior to the dark phase. Compounds were either water soluble or dissolved in up to 3% cremophor (in this case the vehicle also contained cremophor). The pH was adjusted from a minimum of 5.5 to a maximum of 8 depending on the nature of the compound.

As shown in FIGS. 2 and 3, compounds of Formula (I) are useful for decreasing body weight in mice.

### Leptin Assay in Non-Recombinant System

Although well-characterised in recombinant systems (e.g. ObRb-transfected HEK293 cells), where leptin elicits a very marked increase in STAT3 phosphorylation, these systems have often failed to provide an accurate measure of activity of a test compound towards the leptin receptor. It seems that overexpression of the receptor (as well as the possibility for different drugs to act on different parts of the signaling pathway triggered by leptin association with its receptor) results in most cases in the absence of activity of the drugs tested.

The leptin receptor expression in non-recombinant system is often fluctuating and care must be given to identify a system where signal stability remains within experiments. Using such a system, leptin receptor antagonist mimetics could be identified by evaluating their action vs. leptin (see below).

Leptin is produced chiefly in adipose cells, but in humans, mRNA encoding leptin is also present in the placenta. Here, leptin might play an important proliferative role in the microvasculature. The possibility to use this hypothesis in a native cell line was evaluated.

### JEG-3 Protocol

In JEG-3 cells (choriocarcinoma cell line) leptin is able to stimulate proliferation up to 3 fold (Biol. Reprod. (2007) 76: 203-10). Leptin also causes a concentration-dependent increase in [³H]-thymidine incorporation in JEG-3 cells (FIG. 4, maximal effect at 100 nM (EC₅₀=2.1 nM)). The radioactivity incorporated by the cells is an index of their proliferative activity and is measured in counts per minute (CPM) with a liquid scintillation beta counter.

This finding can be applied to test whether a compound is able to either reproduce the effect of leptin on cell proliferation (leptin receptor agonist mimetic) (i.e., a given compound will cause an increase in incorporated [³H]-Thymidine by the cells) or to inhibit the effect of leptin (antagonistic effect) by preventing the leptin-mediated increase in [³H]-thymidine incorporation.

This approach has the advantage of using a non-recombinant system and has reasonable reproducibility and robustness.

### Measurement of Brain Penetration

The test species (rodent) is given a bolus dose of the substrate under investigation, usually via intravenous (IV) or oral (PO) routes. At appropriate time points, blood samples are taken and the resultant plasma extracted and analysed for substrate concentration and, where appropriate, metabolite concentration. At similar time points, animals from another group are sacrificed, brains isolated and the brain surface cleaned. Brain samples are then homogenised, extracted and analysed for substrate concentration and, where appropriate, metabolite concentration. Alternatively, microdialysis probes are implanted into one or more brain regions of the test species and samples collected at appropriate time points for subsequent analysis. This method has the advantage of measuring only extra-cellular substrate concentration. Plasma and brain concentrations are then compared and ratios calculated, either by comparison of averaged concentrations at individual time points, or by calculation of the area-under-the-curve (AUC) of the concentration-time plots.

1. A compound of formula (I) 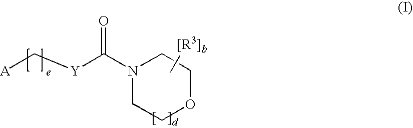
or a pharmaceutically acceptable salt thereof, wherein:
A is selected from 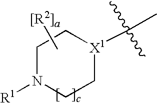
wherein X¹ is N or CH;
and 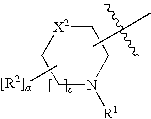
wherein X² is N(R¹), CH(R^{2a}) or O;
R¹ is selected from hydrogen, C₁₋₆-alkyl (unsubstituted or optionally substituted with one or more substituents independently selected from halogen, hydroxy, cyano and C₁₋₆-alkoxy), C₁₋₆-acyl (unsubstituted or optionally substituted with one or more substituents independently selected from halogen, hydroxy and C₁₋₆-alkoxy), phenyl and benzyl (both unsubstituted or optionally substituted with one or more substituents independently selected from halogen, hydroxy, cyano, nitro, CF₃, C₁₋₆-alkyl and C₁₋₆-alkoxy);
each R² and each R³ is independently selected from hydrogen, halogen, hydroxy, C₁₋₆-alkyl (unsubstituted or optionally substituted with one or more substituents independently selected from halogen, hydroxy and C₁₋₆-alkoxy) and C₁₋₆-alkoxy (unsubstituted or optionally substituted with one or more substituents independently selected from halogen, hydroxy and C₁₋₆-alkoxy);
R², is selected from the group consisting of hydrogen and R²;
Y is CH₂, O or N(R⁴);
R⁴ is hydrogen or C₁₋₄-alkyl;
a and b are each independently 0, 1, 2 or 3;
c and d are each independently 0, 1 or 2; and
e is 1, 2 or 3;
with the proviso that the compound is not selected from the group consisting of:
2-(4-piperidinyl)ethyl (2R,6S)-2,6-dimethylmorpholine-4-carboxylate;
N-(4-piperidinylmethyl)-4-morpholinecarboxamide;
4-[1-oxo-3-(4-piperidinyl)propyl]-morpholine;
4-[1-oxo-3-(1-piperazinyl)propyl]-morpholine;
4-[3-[4-(4-chlorophenyl)-1-piperazinyl]-1-oxopropyl]-morpholine;
N-[3-(1-piperazinyl)propyl]-4-morpholinecarboxamide;
2-morpholinylmethyl 2-(hydroxymethyl)-4-morpholinecarboxylate; and
N-[[4-(3,4-dichlorophenyl)methyl]-2-morpholinyl]methyl-4-morpholine-carboxamide.

2. A compound according to claim 1, or a pharmaceutically acceptable salt thereof, wherein Y is O.

3. A compound according to claim 1, or a pharmaceutically acceptable salt thereof, wherein A is

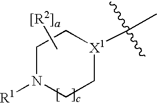

4. A compound according to claim 1, or a pharmaceutically acceptable salt thereof, wherein R¹ is selected from C₁₋₄-alkyl, cyano-C₁₋₄-alkyl, phenyl and benzyl.

5. A compound according to claim 1, or a pharmaceutically acceptable salt thereof, wherein a and b are each independently 0, 1 or 2 and R² and R³, if present, are methyl.

6. A compound according to claim 1, or a pharmaceutically acceptable salt thereof, wherein the compound is of formula (I′) 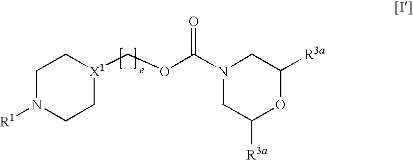
wherein each R^{3a} is hydrogen or R³ and X¹, R¹ and R³ and e are as defined in claim 1.

7. A compound according to claim 6, or a pharmaceutically acceptable salt thereof, wherein both R^{3a} are methyl.

8. The compound according to claim 7, or a pharmaceutically acceptable salt thereof, wherein the two methyl groups are in cis-configuration.

9. A compound according to claim 1, or a pharmaceutically acceptable salt thereof, which is selected from:
(1-benzylpiperidin-4-yl)methyl morpholine-4-carboxylate;
2-[4-(cyanomethyl)piperazin-1-yl]ethyl (2R,6S)-2,6-dimethylmorpholine-4-carboxylate; and
2-(4-methylpiperazin-1-yl)ethyl (2R,6S)-2,6-dimethylmorpholine-4-carboxylate
and pharmaceutically acceptable salts thereof.

10. A pharmaceutical formulation, containing a compound according to claim 1, or a pharmaceutically acceptable salt thereof, as an active ingredient, in combination with a pharmaceutically acceptable diluent or carrier.

11. A method for treatment or prevention of conditions or diseases associated with weight gain, which comprises administering to a mammal, including man, in need of such treatment an effective amount of a compound according to claim 1, or a pharmaceutically acceptable salt thereof.

12. The method according to claim 11, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

13. A method for treatment or prevention of severe weight loss, dysmenorrhea, amenorrhea, female infertility or immunodeficiency, or the treatment of wound healing, which comprises administering to a mammal, including man, in need of such treatment an effective amount of a compound according to claim 1, or a pharmaceutically acceptable salt thereof.

14. A method for treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis, which comprises administering to a mammal, including man, in need of such treatment an effective amount of a compound according to claim 1, or a pharmaceutically acceptable salt thereof.

15. A method for inhibition of angiogenesis, which comprises administering to a mammal, including man, in need of such treatment an effective amount of a compound according to claim 1, or a pharmaceutically acceptable salt thereof.

16. A process for the preparation of a compound of claim 1, comprising:
(a) reacting a compound of formula (II): 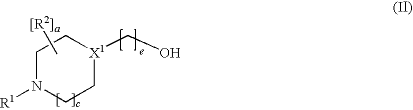
wherein X¹, R¹, R², a, c and e are as defined in claim 1,
with 4-nitrophenyl chloroformate or bis-(4-nitrophenyl)carbonate in the presence of a suitable base (such as NMM) in a suitable solvent (such as DCM), at −10 to 40° C., to form a compound of formula (III): 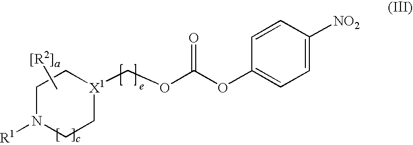
(b) reacting the compound of formula (III) with a compound of formula (IV): 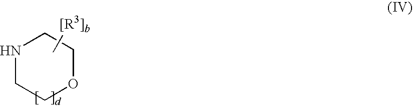
wherein R³, b and d are as defined in claim 1,
in the presence of a suitable base, (such as DIPEA or DMAP), in a suitable solvent (such as DMF), at −10 to 40° C., to obtain a compound of formula (I); and
(c) optionally, in one or several steps transforming a compound of formula (I) into another compound of formula (I).
